(19) United States
(12) Patent Application Publication
(10) Pub. No.: US 2013/0196942 A1
(43) Pub. Date: Aug. 1, 2013

(54) Composition for Skin Treatment

(71) Applicant: Kenneth James, West Berkshire (GB)

(72) Inventor: Kenneth James, West Berkshire (GB)

(73) Assignee: Bio Skincare Limited, London (EC)

(21) Appl. No.: 13/877,530

(22) PCT Filed: Oct. 5, 2011
(86) PCT No.: PCT/GB2011/051893

§ 371 Date: Apr. 3, 2013

(30) Foreign Application Priority Data
Oct. 6, 2010 (GB) 1016834.2
Jun. 23, 2011 (GB) 1110653.1

(51) Int. Cl. A61K 31/7016

(52) U.S. Cl. CPC A61K 31/7016
USPC 514/53

(57) ABSTRACT

A composition comprising from 10 to 15% by weight of sucrose palmitate. The composition can be used to treat eczema.

The present invention relates to a semi-solid composition comprising a sucrose fatty acid ester and its use in the treatment of skin conditions.

Skin conditions such as eczema afflict a significant proportion of the population. A recent study in the United States of America found over 17% of the population exhibited at least one eczematous symptom and empirically defined eczema was found in just over 10% of the population (Dermatitis. 2007; 18(2):82-91). Many of the existing treatments for severe forms of eczema use active ingredients such as steroids which have undesirable side effects such as thinning of the skin. Accordingly, there is significant interest in identifying new alternative treatments for eczema.

The use of fatty acids, fatty acid salts and sucrose esters in cosmetic compositions and compositions that come into contact with the skin is known.

U.S. Pat. No. 2,733,252 discloses a process for the preparation of the fatty acid esters of lactylic acid and salts thereof in a commercial environment.

U.S. Pat. Nos. 3,098,795 and 4,422,952 disclose the use of fatty acid esters as emulsifiers in a composition.

U.S. Pat. Nos. 3,896,238, 4,150,114 and 4,046,886 disclose the use of a sucrose ester in combination with an alkyl sulfoxide or phosphine oxide in compositions for enhancing the penetration of pharmacologically active agents into the skin. Preferred sucrose esters include mono- and diacyl esters wherein the acyl substituents contain 8 to 20 carbon atoms with sucrose monooleate the most preferred.

U.S. Pat. No. 4,822,601 discloses a cosmetic base composition exhibiting therapeutic properties which comprises an acyl fatty acid lactylate ester or alkali metal salt thereof, a sucrose fatty acid ester and a solvent.

U.S. Pat. No. 4,990,501 discloses compositions for application to human and animal tissue surfaces e.g. skin. The compositions comprise an aqueous medium and a film forming component such as a sucrose ester that is at least partially soluble in water.

Surprisingly it has been found that compositions comprising a non-water-soluble sucrose fatty acid ester as the therapeutic ingredient are effective for use in the treatment of eczema. In particular, these compositions may consist essentially of the non-water-soluble sucrose fatty acid ester as the only therapeutic ingredient. The composition may include further ingredients to improve its properties and ease of application.

Accordingly, the present invention provides a composition comprising from 1 to 50% by weight of a non-water-soluble sucrose fatty acid ester. Preferably the composition comprises from 2 to 40%, more preferably from 5 to 30%, more preferably from 7.5 to 25%, most preferably from 10 to 15% of the non-water-soluble sucrose fatty acid ester.

In this document, the term ‘non-water-soluble’, in relation to a sucrose ester, means that a mixture of 90% water and 10% of the sucrose ester, by weight, at 20° C. does not form a single phase.

The non-water-soluble sucrose fatty acid ester is typically an ester of a saturated acid having from 14 to 20 carbon atoms. Suitable fatty acid esters include sucrose myristate, sucrose palmitate and sucrose stearate. The fatty acid ester may comprise a mono acid ester, a di-acid ester, a tri-acid ester, a poly-acid ester or a mixture thereof. Sucrose palmitate is a preferred non-water-soluble sucrose fatty acid ester for use in the present invention. Sucrose palmitate is preferably either sucrose monopalmitate, sucrose dipalmitate or a mixture thereof.

In a preferred embodiment of the present invention the composition comprises from 1 to 50% by weight of a non-water-soluble sucrose fatty acid ester wherein the sucrose ester comprises at least about 70% by weight of a sucrose ester of one organic acid. For example the sucrose ester is nominally sucrose palmitate, and is made with an organic acid comprising at least about 70% palmitic acid. Alternatively the sucrose ester is nominally sucrose stearate, and is made with an organic acid that is at least about 70% stearic acid; or the sucrose ester is nominally sucrose myristate made with an organic acid that is at least about 70% myristic acid.

In a preferred embodiment of the present invention the composition comprises from 7.5 to 25%, by weight, more preferably from 10 to 15% of sucrose palmitate. Preferably the therapeutic ingredient of the composition consists essentially of sucrose palmitate.

In a preferred embodiment of the present invention the composition comprises a non-water-soluble sucrose fatty acid ester in combination with cosmetically and/or pharmaceutically acceptable excipients and diluents. Surprisingly, it has been found that neither a water solubilising ester nor a film forming agent is required in the composition of the present invention. Typical further components of the composition of the present invention include oils and waxes, components to improve the skin feel of the resulting composition, and preservative. Preferably the composition comprises more non-water-soluble sucrose fatty acid ester than oil and wax for example, twice as much non-water-soluble sucrose fatty acid ester as oils and waxes.

In a preferred embodiment of the present invention the composition is semi-solid.

In a particularly preferred embodiment of the present invention the semi-solid composition comprises emulsifier, oil and/or wax and from 10 to 15% by weight of sucrose palmitate, where the composition comprises a greater proportion of sucrose palmitate than of oil and/or wax and wherein the total amount of emulsifier in the composition is up to 20% by weight of the composition.

In a particularly preferred embodiment, sucrose palmitate is the only sucrose ester in the composition.

The composition forms an emulsion of oily components in water. Therefore preferred emulsifiers are those which have a hydrophilic lipophilic balance (HLB) of from 12 to 18, preferably from 14 to 18. Typical emulsifiers include sorbitan fatty acid esters, ethoxylated derivatives of sorbitan fatty acid esters, alkyl polyglucosides and lactitol fatty acid ester, preferably sorbitan fatty acid esters and ethoxylated derivatives of sorbitan fatty acid esters. In addition, sucrose fatty acid esters typically have an HLB in the range 12 to 18 and are considered to be emulsifiers of the present invention.

The composition of the present invention is a stable emulsion of oil in water. The composition is a semi-solid composition such as a cream and it is not in the form of a liquid.

In a preferred embodiment of the present invention, the composition comprises up to 15% emulsifier.

The oils and waxes of the present invention include oils and waxes such as refined coconut oil, fractionated coconut oil, light mineral oil, stringy petrolatum, beeswax, beeswax substitute, lanolin, tea tree oil, lavender oil, eucalyptus oil, rosemary oil and apricot kernel oil. In a preferred embodiment the composition of the present invention comprises a ratio of sucrose palmitate to oil and/or wax of 3:1, more preferably 3:2, more preferably 2:1.

In a particularly preferred embodiment of the present invention the composition comprises up to 20% of an emulsifier with an HLB of 12-18.

Surprisingly, it has been found that the compositions of the present invention are effective in the treatment of eczema. When applied to patients, an improvement in their skin condition was visible within as little as 1 to 2 days. Accordingly, the present invention provides a composition of the present invention for use in the treatment of eczema. In particular, the present invention provides a composition of the present invention where the active ingredient consists essentially of one or more non-water-soluble fatty acid esters for use in the treatment of eczema. In a preferred embodiment the present invention provides a composition where the non-water-soluble sucrose fatty acid ester consists essentially of sucrose palmitate for use in the treatment of eczema.

The present invention also provides a method of treating eczema, the method comprising administering to a subject in need of such treatment an effective amount of a non-water-soluble sucrose fatty acid ester, such as a composition of the present invention. The present invention also provides the use of a non-water-soluble sucrose fatty acid ester in the preparation of a medicament for the treatment of eczema.

It is known that barrier creams are useful in the treatment of eczema. Although a non-water-soluble sucrose fatty acid ester does not form a visible film, it is thought that the composition of the invention works by forming a barrier on the skin. Such a barrier compensates when the skin's barrier function is compromised, allowing healing of the damaged, compromised or diseased skin to take place.

It has further been found that the compositions of the present invention are effective in the treatment of ulcers, wounds, scar tissue and nappy rash. Accordingly the present invention provides the use of a composition of the present invention in the treatment of ulcers, wounds, scar tissue or nappy rash. In particular, the present invention provides the use of a composition of the present invention where the active ingredient consists essentially of one or more non-water-soluble fatty acid esters in the treatment of ulcers, wounds, scar tissue or nappy rash. In a preferred embodiment the present invention provides the use of a composition where the non-water-soluble sucrose fatty acid ester consists essentially of sucrose palmitate in the treatment of ulcers, wounds, scar tissue or nappy rash.

The present invention also provides a method of treating ulcers, wounds, scar tissue or nappy rash, the method comprising administering to a subject in need of such treatment an effective amount of a non-water-soluble sucrose fatty acid ester such as a composition of the present invention. The present invention also provides the use of a non-water-soluble sucrose fatty acid ester in the preparation of a medicament for the treatment of ulcers, wounds, scar tissue or nappy rash.

It has further been found that the compositions comprising sucrose palmitate, and in particular compositions of the present invention are effective in the treatment of sunburn, cracked heels and athlete's foot. Accordingly the present invention provides the use of a composition comprising sucrose palmitate, in particular a composition of the present invention in the treatment of sunburn, cracked heels and athlete's foot. In particular, the present invention provides the use of a composition comprising sucrose palmitate, in particular a composition of the present invention where the active ingredient consists essentially of one or more non-water-soluble fatty acid esters in the treatment of sunburn, cracked heels and athlete's foot. In a preferred embodiment the present invention provides the use of a composition where the non-water-soluble sucrose fatty acid ester consists essentially of sucrose palmitate in the treatment of sunburn, cracked heels and athlete's foot.

The present invention also provides a method of treating sunburn, cracked heels and athlete's foot, the method comprising administering to a subject in need of such treatment an effective amount of a non-water-soluble sucrose fatty acid ester such as a composition comprising sucrose palmitate or a composition of the present invention. The present invention also provides the use of a non-water-soluble sucrose fatty acid ester in the preparation of a medicament for the treatment of sunburn, cracked heels and athlete's foot.

The following illustrative examples and figures relating to formulations made in accordance with the present invention are intended to illustrate typical compositions and are not intended to be limiting on the scope of the invention. All materials used in the formulations are commercially available.

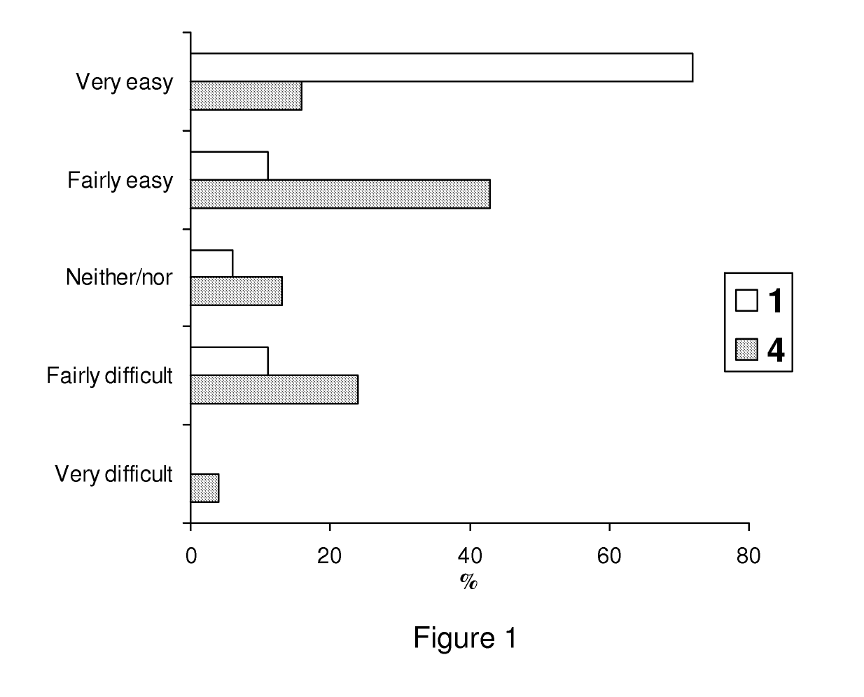

FIGS. 1 to 3 display the data obtained in example 16 in graph form.

FIG. 1 compares the ease of applying the creams of formulations 1 and 4.

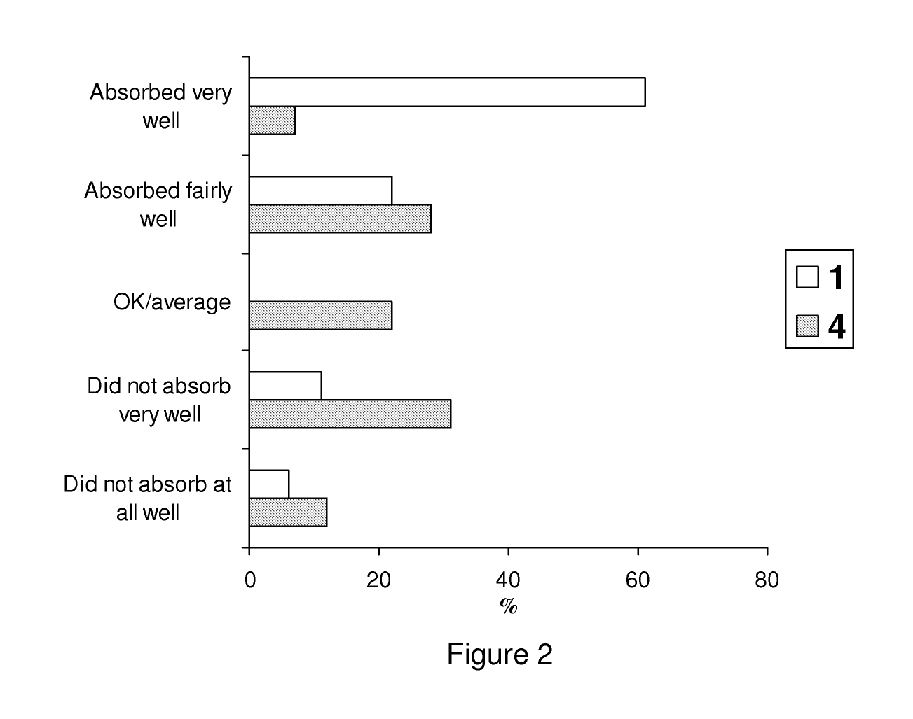

FIG. 2 compares the absorption of the creams of formulations 1 and 4.

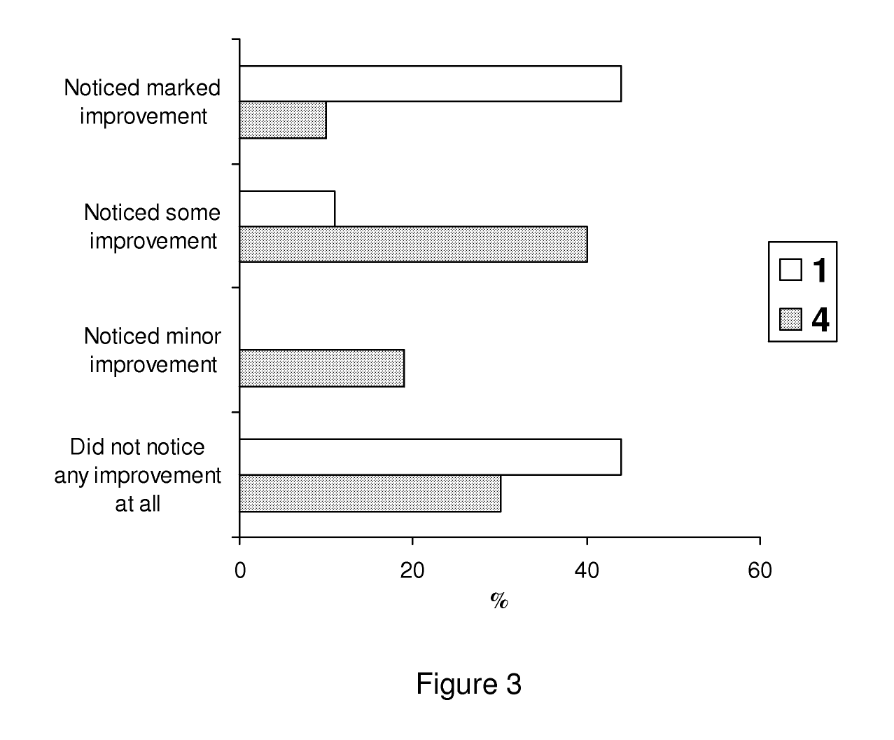

FIG. 3 compares the improvement noticed by respondents to their eczema when using formulations 1 or 4.

### EXAMPLE 1

Formulation 1

| Ingredient | Percent (by weight) |
|---|---|
| Sucrose mono/dipalmitate | 12 |
| Oils and waxes | 4 |
| (as Beeswax 2%, Apricot kernel oil 1% and | |
| Fractionated coconut oil 1%) | |
| Isopropyl myristate | 3 |
| Cetyl alcohol | 2 |
| Calcium stearate | 1 |
| Preservative | 0.3 |
| Soft water | 77.7 |

### EXAMPLE 2

Formulation 2

| Ingredient | Percent (by weight) |
|---|---|
| Sucrose mono/dipalmitate | 10 |
| Oils and waxes | 4 |
| (as Beeswax 2%, Apricot kernel oil 1% and | |
| Fractionated coconut oil 1%) | |
| Isopropyl myristate | 3 |
| Calcium stearate | 2 |
| Cetyl alcohol | 3 |
| Preservative | 0.3 |
| Soft water | 77.7 |

The following illustrative examples relating to efficacy are in accordance with the present invention and are not intended to be limiting on the scope of the invention.

### EXAMPLE 3

The composition of Formulation 1 was used for an in-home placement study conducted on a sample of adults suffering from mild eczema. Respondents were given a sample tub of the cream of formulation 1 and were instructed to apply the cream to dry/eczematous skin twice per day as they would their normal emollient cream. A diary sheet was placed with the cream for respondents to record their experience of the product during the trial period. Following a placement period of approximately 2 weeks a follow-up interview was conducted to assess opinions of the product.

58% of respondents noted an improvement in their mild eczema. 70% of these noted an improvement after 1-2 days, 20% after 3-4 days and 10% after 5-6 days.

### EXAMPLE 4

Formulation 3

| Ingredient | Percent (by weight) |
|---|---|
| Sucrose mono/dipalmitate | 15 |
| Beeswax | 2 |
| Calcium stearate | 4 |
| Titanium dioxide | 0.08 |
| Glycerol | 1-16 |
| Mono/diglycerides | 9 |
| Cetyl alcohol | 2 |
| Preservative | 0.3 |
| Soft water | 67.32-51.9 |

The composition of Formulation 3 was used in selective cases on scar tissue and found to be efficacious in hastening resolution of scarring and reducing its eventual severity. The formulation was also noted to have considerable soothing properties.

### EXAMPLE 5

Formulation 4

| Ingredient | Percent (by weight) |
|---|---|
| Sucrose mono/dipalmitate | 15 |
| Mono/diglycerides | 9 |
| Cetyl alcohol | 2 |
| Beeswax | 2 |
| Glycerol | 0.2 |
| Calcium stearate | 4 |
| Titanium dioxide | 0.08 |
| Preservative | 0.3 |
| Water | 67.67 |

The composition of Formulation 4 was used in selective cases on children under 3 years of age with nappy rash. The composition was reported by mothers to be efficacious in hastening the resolution of nappy rash.

In each of formulations 1-4 the pH of the resulting formulation is typically in the range 4.5-8.0. This pH is preferred as it encompasses the skins natural pH level, and is therefore less likely to cause skin irritation.

### EXAMPLE 6

The composition of Formulation 4 was used in selected cases on cuts, one cut being particularly severe, and in all cases promoted healing within two to three days.

### EXAMPLE 7

The composition of Formulation 4 was used to treat lesions from burns. It alleviated pain, prevented blistering and accelerated healing.

### EXAMPLE 8

The composition of Formulation 1 was used to treat athlete's foot in an adult where skin had flaked off and blisters had formed. The composition was applied once and relieved the itching associated with the infected area. The skin healed without further symptoms and there was no recurrence of the infection at that site during the subsequent monitoring period of over two months.

### EXAMPLE 9

The composition of Formulation 1 was used to treat athlete's foot in a further adult (other than in Example 8). The composition was applied several times over a period of a few days and the area healed within a week.

### EXAMPLE 10

The composition of Formulation 4 was applied to the hands of an adult to alleviate the pain from burns acquired during cooking. The patient reported a significant reduction in pain and the burn healed fast and without blistering.

### EXAMPLE 11

The composition of Formulation 4 was applied to the cracked skin on the heels of an adult. The pain and cracking were significantly reduced.

### EXAMPLE 12

The composition of formulation 1 was applied to the skin of an adult who had sunburn. The formulation was found to alleviate the sunburn.

### EXAMPLE 13

The composition of formulation 1 was applied to the sunburnt skin of an adult. The skin was exposed to sunburn when swimming in a swimming pool in the Caribbean in April for at least 1 hour. The composition significantly reduced the pain of the sunburn and repeated applications for the next 4 days prevented the skin from peeling and flaking.

### EXAMPLE 14

The composition of Formulation 1 was applied to a wound to the knee in an adult caused by contact with a sea urchin while diving. The composition was applied after the sea urchin spines had been removed (by treatment with vinegar). The composition immediately relieved the pain and itchiness and the wound healed quickly.

### EXAMPLE 15

The composition of Formulation 1 was used by an adult who had suffered from serious eczema for over 30 years. The patient found that the composition made a significant difference to their eczema.

### EXAMPLE 16

A comparative study was carried out to compare the composition of formulation 1 with the composition of formulation 4.

Formulation 4

| | |
|---|---|
| Sucrose mono/dipalmitate | 15 |
| Mono/diglycerides | 9 |
| Cetyl alcohol | 2 |
| Beeswax | 2 |
| Glycerol | 0.2 |
| Calcium stearate | 4 |
| Titanium dioxide | 0.08 |
| Preservative | 0.3 |
| Water | 67.67 |

An in-home placement study was carried out for each of these formulations. 100 respondents were given a tub of Formulation 4 for about 2 weeks and a diary sheet to record their usage and perceptions of the cream.

Subsequently the respondents were given a tub of Formulation 1 for about 2 weeks and again given a diary sheet. All of the respondents suffered from some form of eczema.

The respondents found it significantly easier to apply the composition of formulation 1 than formulation 4 and the composition of formulation 1 was perceived by the respondents to be better absorbed into the skin. The results can be seen in FIGS. 1 and 2. In addition, more of the respondents noticed a marked improvement in their eczema using the composition of formulation 1. In contrast, most respondents noticed at best a ‘some’ improvement in their eczema when using formulation 4. This is shown in FIG. 3.

Overall the composition of formulation 1 was perceived to be a markedly preferred composition compared to formulation 4.

Three-quarters of the respondents rated the composition of formulation 1 as ‘very easy’ to apply (vs 16% for the comparative formulation)

Over four out of five rated it as ‘absorbing well’ (vs 35% for the comparative formulation). 44% of respondents rated the skinfeel as ‘very pleasant’ (vs 11% for formulation 4).

The overall opinion of and quality of formulation 1 were also better. 39% rated formulation 1 as ‘very good’ overall (vs 8% for formulation 4)
44% rated the overall quality of formulation 1 as ‘very good’ (vs 5% for formulation 4)

The perceived effectiveness of formulation 1 was also higher than that for formulation 4.

56% rated it as ‘very effective’ (vs 11% for formulation 4). 44% noted a ‘marked improvement’ after use (vs 10% for formulation 4).

The fact that Formulation 1 was preferred to formulation 4 in qualities such as absorption, overall quality and ease of application is very significant. It has been noted in the literature that the compliance of patients using creams is heavily linked to their perception of the composition. Where patients do not like the feel of the composition, they are reluctant to apply the composition in sufficient quantity or often enough to achieve the available benefit. It is therefore very important to formulate a skin composition so as to appeal to the users.

What is claimed:

1. A semi-solid composition comprising emulsifier, oil and/or wax and from 10 to 15% by weight of sucrose palmitate, where the composition comprises a greater proportion of sucrose palmitate than of oil and/or wax and wherein the total amount of emulsifier in the composition is up to 20% of the composition.

2. A composition according to claim 1 wherein the composition is a cream.

3. A composition according to claim 1 wherein the composition comprises more than one emulsifier.

4. A composition according to claim 1 wherein the composition comprises up to 15% of emulsifier.

5. A composition according to claim 1 wherein the ratio of sucrose palmitate to oil and/or wax is at most 2:1.

6. A method comprising carrying out therapy with the composition of claim 1.

7. A method comprising treating with the composition of claim 1.

8. A method comprising treating ulcers, wounds, scar tissue or nappy rash with a composition comprising sucrose palmitate.

9. A method comprising treating ulcers, wounds, scar tissue or nappy rash with the composition according to claim 1.

10. A method comprising treating sunburn, cracked heels or athletes foot with a composition comprising sucrose palmitate.

11. A method comprising treating sunburn, cracked heels or athletes foot with the composition according to claim 1.
